(19) United States
(12) Patent Application Publication
(10) Pub. No.: US 2013/0310640 A1
(43) Pub. Date: Nov. 21, 2013

(54) FLEXIBLE GASTROINTESTINAL VIDEOENDOSCOPE

(71) Applicants: Kiyoshi Hashiba, Sao Paulo (BR); Dorival Filho Zito, Sao Paulo (BR); Pablo Rodrigo de Siqueira, Sao Paulo (BR); Helio Shigueki Ozawa, Sao Paulo (BR)

(72) Inventors: Kiyoshi Hashiba, Sao Paulo (BR); Dorival Filho Zito, Sao Paulo (BR); Pablo Rodrigo de Siqueira, Sao Paulo (BR); Helio Shigueki Ozawa, Sao Paulo (BR)

(21) Appl. No.: 13/879,760

(22) PCT Filed: Oct. 19, 2011
(86) PCT No.: PCT/BR11/00380

§ 371 Date: May 16, 2013

(30) Foreign Application Priority Data
Oct. 19, 2010 (BR) PI1003817-5

(51) Int. Cl. A61B 1/005; A61B 1/00; A61B 1/273; A61B 1/04

(52) U.S. Cl. CPC A61B 1/0057; A61B 1/04; A61B 1/00009; A61B 1/00052; A61B 1/2736
USPC 600/103; 600/109

(57) ABSTRACT

The invention relates to a compact videoendoscope, which has additional movements on its tip, allowing better utilization of the movements performed in the distal segment of the device. The videoendoscope according to the invention also provides, in addition, an image processor and monitor incorporated to the handle, or proximal part of the device, with light originating from the device itself through LEDs placed at the distal tip.

### FIELD OF THE INVENTION

The present invention applies to the field of medicine, more specifically to the field of endoscopy. The invention relates to a compact version of a videoendoscope, capable of an additional movement in its tip, allowing greater utilization in the distal segment of the device.

The videoendoscope according to the invention comprises, simultaneously, an image processor and a display incorporated to the handle or to the proximal part of the device with light originating from the device itself through LEDs placed at the tip thereof.

### BACKGROUND

Endoscopy was born around 1870, when Kussmaul hired a sword swallower to swallow a tube, thereby allowing examination of his esophagus.

After that, for a long time a metal tube was employed for said purpose; subsequently a tube was developed (Schindler in 1932) in which lenses were introduced, allowing limited curvature movements. This endoscope was named “semi flexible”.

Changes came in 1956 when it was attempted to use glass fibers to conduct images (Hirschowitz); however, the pictures had a poor quality and the device was unwieldy.

In 1962 Uji used a miniaturized camera (film size between 5 and 7 mm), allowing documenting the inner face of the stomach, although not providing a direct view for the operator.

In 1963 the use of glass fibers to allow direct vision was introduced, the illumination being provided by a light bulb.

From 1965 onward the devices were provided with the light of an external lamp being conducted by glass fibers. The image was also conducted to an optical element placed at the proximal segment of the device.

Further evolution of the endoscopes came when the image began to be conducted electronically from a chip placed at the distal end of the device.

The endoscopes comprised a duct connected to the proximal segment, enclosing the glass fibers, an air tube to inflate the organ being examined and another tube connected to an aspirator to remove the same air as well as secretions. A container with water was linked on the path of the air injection, which by Venturi effect allowed squirting water to wash the lens of the device.

Of course this duct was also connected to a light source and an image processor that received the image and displayed it on a monitor. Thus, the examiner looked an image at a monitor which received the images from the distal lens, captured by the chip and conducted electronically.

Thus resulted the known form of the current endoscope which, for obvious reasons, is called a videoendoscope.

The videoendoscope set, with all the advances that have been introduced, is still quite expensive in Brazilian market (about 90,000 USD).

Based on the photo and video cameras, which also process the image provided from a lens linked to a chip, and in the lighting with LED devices, a type of endoscope was created which was similar to those already existing for non-medical purposes, endowed with some flexibility, which are called borescopes.

As seen in the figures related to the prior art (FIGS. 1 and 2), existing endoscopes have a flexible part (3), which curves in a wide radius, covered by plastic tubing. In general, the extreme portion (4) of a known endoscope comprises a more flexible part which allows a tighter curvature, having a distal rigid tip of about 2 cm, into which fit the lens with the chip (5), the ends of the illumination fibers (7 and 7′), the working channels (8), the insufflation and aspiration tubes (6).

### SUMMARY OF THE INVENTION

The main purpose of the present invention is to provide a compact videoendoscope, which allows a broader range of procedures than conventional endoscopes, enabling greater operational range, thereby allowing a single device to replace different types of endoscopes.

Another purpose of the present invention is to provide a compact videoendoscope at a lower cost than the devices currently used by endoscopy specialists and clinics.

It is known that endoscopy is currently an important tool for diagnosis, besides playing an important therapeutic role.

Thus, it is yet another purpose of the invention to provide a videoendoscope that can be widely used in economically disadvantaged regions, minimizing the cost of health treatments in the field of gastroenterology, in which endoscopy has a primary function (it is said that “today nothing is done in gastroenterology without endoscopy”).

### General Description of the Invention

The videoendoscope according to the invention has in its distal tip particular features specially developed for the desired aims.

The above mentioned purposes are achieved by the invention by the provision of a videoendoscope with features that allow additional movements of its distal tip, having, in addition, coupled to it, an image processor and a monitor in the proximal part of the device, with light originated on the device itself through LEDs located on its end.

The fact that the endoscope according to the present invention also possesses an imaging device and a monitor arranged at the proximal end contributes to reducing the cost of the equipment.

The aforementioned additional movement is the result of a greater range of angular curvature of the tip of the videoendoscope based on the invention in its last 2 cm, which produces technical effects not present in conventional devices.

The videoendoscope according to the invention enables new procedures in biliary and pancreatic ducts that currently are performed only with specific devices (called duodenoscopes).

Thus, it is possible to suppose that the videoendoscope according to the invention will come to perform an ample range of procedures nowadays requiring several conventional devices, “saving up” a device, so to speak.

### BRIEF DESCRIPTION OF THE FIGURES

Further features and advantages of the invention will become more apparent from the description of a preferred embodiment, given by way of example and not by limitation, as well as the figures which refer to it, listed below:

FIGS. 1 and 2 schematically illustrate a prior art endoscope.

FIG. 3 schematically illustrates the flexible rubber cover (10) and steel mesh (11) that cover and protect the entire insertion tube.

FIG. 4 schematically illustrates the vertebra (12), two views of the attachment point of the control cables (13) and the control cables of the videoendoscope (14).

FIG. 5 schematically illustrates the metallic appendix and head (15) with respective openings of the working channel, the lighting and lens, the tension spring (16), and pivoting mechanism (17), with its lateral and upper views of the first and second segments (17a and 17b) and of the pivot (17c), as well as the movements of the second segment (17b) upward and downward (up-down).

FIG. 6 schematically illustrates the components assembly.

FIG. 7 illustrates, schematically, in an internal view, the movement (down) from the distal tip of the videoendoscope.

FIG. 8 schematically illustrates, in an external view, the movement (down) from the distal tip of the videoendoscope.

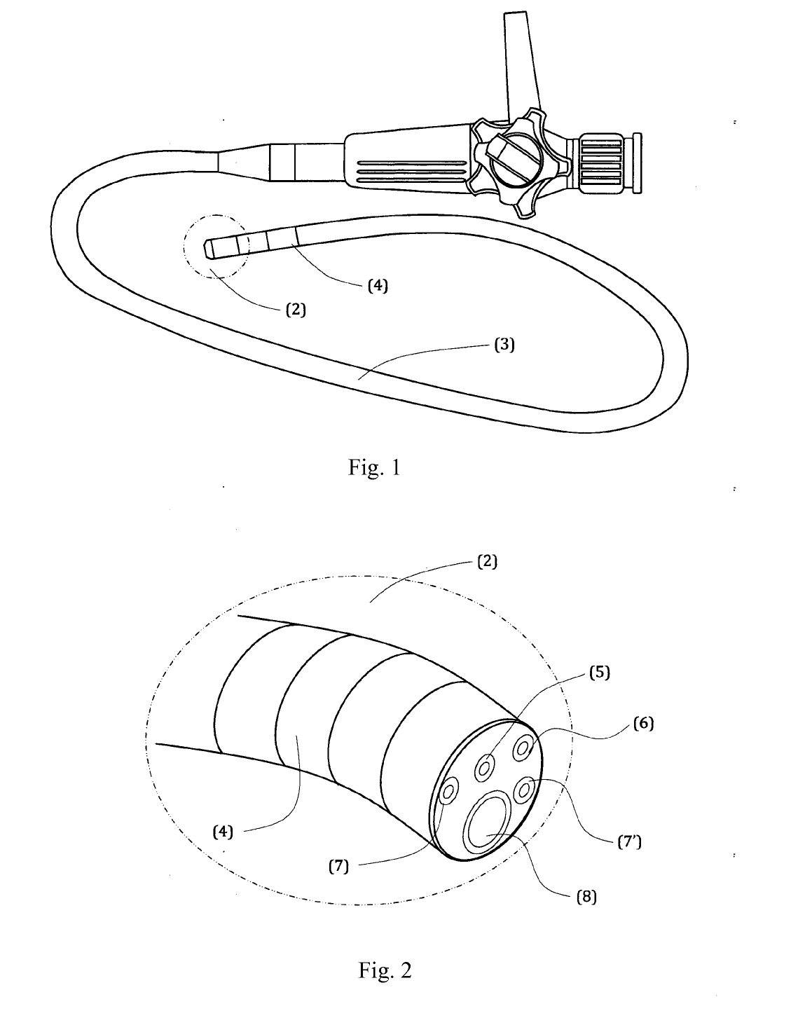

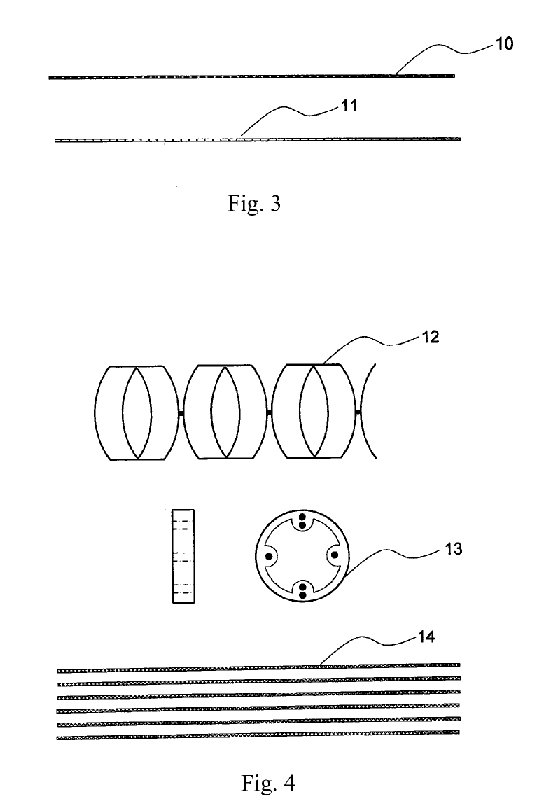

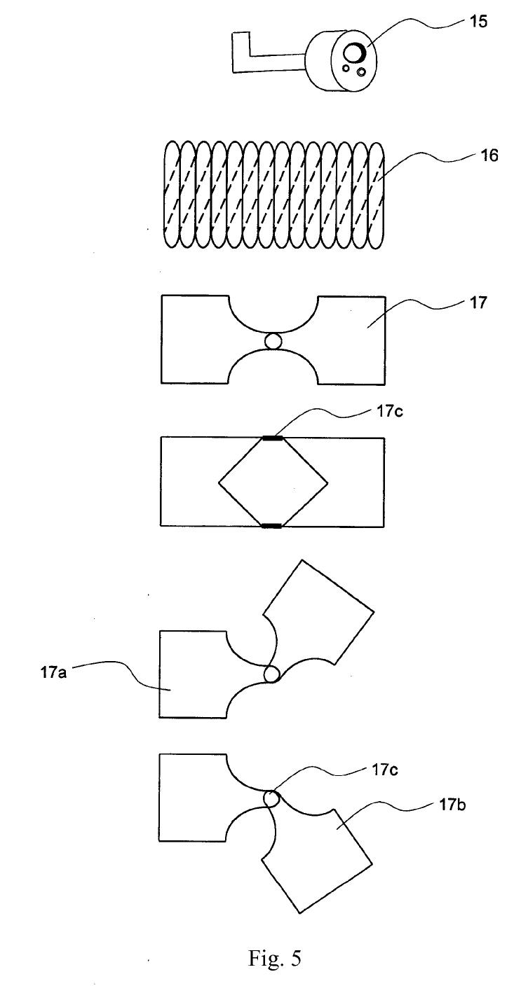

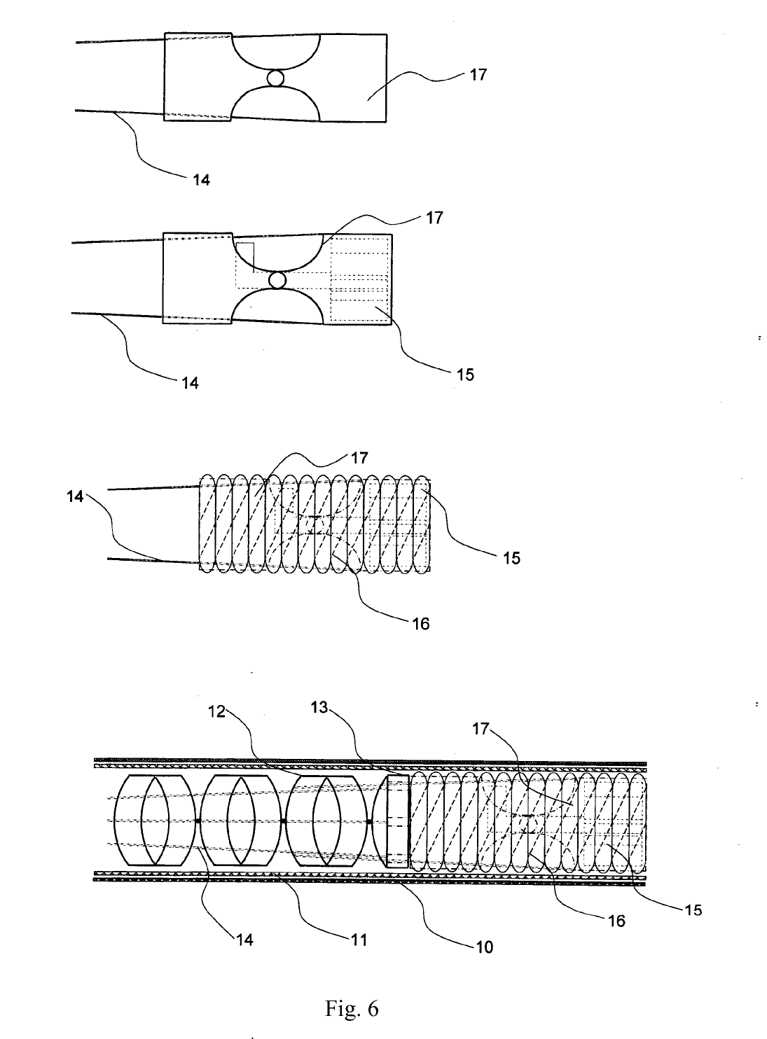

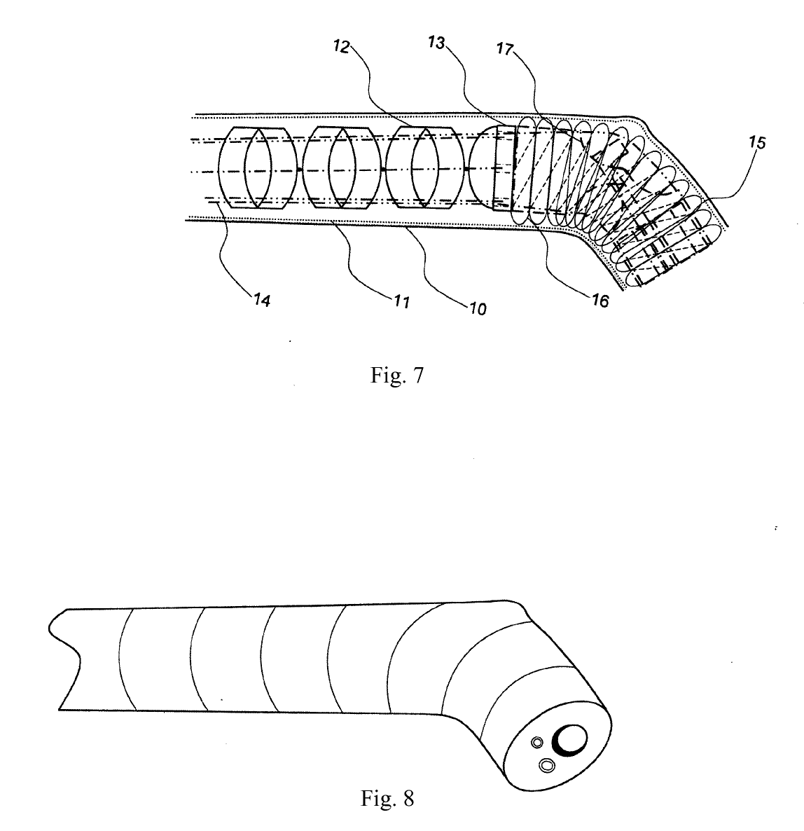

### DETAILED DESCRIPTION OF AN EMBODIMENT OF THE INVENTION

The videoendoscope proposed by the invention has in the interior of its distal end a series of articulated rings, or vertebrae (12), six control cables (14), four of them being fixed at the attachment point (13) that controls movements upward, downward, to the left and to the right of the insertion tube.

A pivoting mechanism (17), completely hollow, is split into two segments (17a and 17b) joined by pivots (17c) that do not cross the entire hollow area of the mechanism.

In the back part of its first segment (17a) there is the attachment point (13), and in the second segment (17b) the two control cables (14) are connected, which pass beyond the attachment point (13) and are fixed in the back part of the second segment (17b) of the pivoting mechanism, thus allowing two additional movements, upwards and downwards.

Attached to the front part of the second segment (17b) of the pivoting mechanism is the metallic appendix and head (15), whereto the control chip (appendix) as well as the openings to the working, lighting and lens (head) channels are attached.

A tension spring (16) covers said pivoting mechanism (17), preventing the steel mesh (11) and flexible hose of the working channel from entering the existing gap between the first and second segments (17a and 17b) of the pivoting mechanism.

These features allow performing procedures in areas not easily reached, as well as procedures that are not performed by conventional endoscopes.

In one embodiment of the invention, the distal end of the insertion tube of the videoendoscope will allow two additional movements (upward and downward), enabling a better view, such features not being present in known endoscopes.

1. FLEXIBLE GASTROINTESTINAL VIDEOENDOSCOPE, characterized in that the insertion tube, in its distal end, has a last segment articulated with pivoting movements.

2. FLEXIBLE GASTROINTESTINAL VIDEOENDOSCOPE, characterized in that it is provided in its distal end, with a pivoting mechanism (17).

3. FLEXIBLE GASTROINTESTINAL VIDEOENDOSCOPE, as claimed in claim 2, characterized in that the pivoting mechanism (17) is totally hollow and divided into two segments (17a and 17b), joined by pivots (17c) which do not pass through the hollow space of the mechanism.

4. FLEXIBLE GASTROINTESTINAL VIDEOENDOSCOPE, as claimed in claim 3, characterized in that the back part of the first segment (17a) of said pivoting mechanism is fitted with the attachment point of the cables (13) that control the movements of the distal tip of the insertion tube, said movements being in two mutually orthogonal planes.

5. FLEXIBLE GASTROINTESTINAL VIDEOENDOSCOPE, as claimed in claim 3, characterized in that, to the second segment (17b) of the pivoting mechanism, two control cables (14) are connected, which pass beyond said attachment point (13) and are fixed on the posterior part of said second segment (17b) of the pivoting mechanism, allowing two additional and independent movements of the distal end.

6. FLEXIBLE GASTROINTESTINAL VIDEOENDOSCOPE, as claimed in claim 3, characterized in that, to the front part of the second segment (17b) of the pivoting mechanism the metallic appendix and head (15) is attached, wherein the control chip and openings for the working, lighting and lens channels are arranged.

7. FLEXIBLE GASTROINTESTINAL VIDEOENDOSCOPE, as claimed in claim 2, characterized in that said pivoting mechanism (17) is surrounded by a tension spring (16).

8. FLEXIBLE GASTROINTESTINAL VIDEOENDOSCOPE, as claimed in any of the preceding claims, further characterized by having an image processor and a monitor coupled in the proximal part of the device.
